(19) United States
(12) Patent Application Publication
(10) Pub. No.: US 2005/0143750 A1
(43) Pub. Date: Jun. 30, 2005

(54) IOL inserter plunger

(76) Inventor: Edward Vaquero, Fairport, NY (US)

Correspondence Address: Bausch & Lomb Incorporated, One Bausch & Lomb Place, Rochester, NY, 14604-2701, US

(21) Appl. No.: 11/020,879

(22) Filed: Dec. 22, 2004

Related U.S. Application Data
(60) Provisional application No. 60/533,405, filed on Dec. 30, 2003.

(51) Int. Cl. A61F 9/00

(52) U.S. Cl. 606/107

(57) ABSTRACT

A plunger for an IOL inserter includes a recess defined between the plunger tip and a curvilinear ramp for positioning the trailing haptic of an IOL during IOL delivery through the inserter and into an eye.

### RELATED APPLICATION

This application claims the benefit under 35 USC §119(e) of prior U.S. Provisional Application Ser. No. 60/533,405 filed Dec. 30, 2003.

### BACKGROUND OF THE INVENTION

The present invention relates to ophthalmic surgical devices. More particularly, the present invention relates to a device for inserting an intraocular lens (IOL) into an eye.

IOLs are artificial lenses used to replace the natural crystalline lens of the eye when the natural lens has cataracts or is otherwise diseased. IOLs are also sometimes implanted into an eye to correct refractive errors of the eye in which case the natural lens may remain in the eye together with the implanted IOL. The IOL may be placed in either the posterior chamber or anterior chamber of the eye. IOLs come in a variety of configurations and materials. Some common IOL styles include the so-called open-looped haptics which include the three-piece type having an optic and two haptics attached to and extending from the optic; the one-piece type wherein the optic and haptics are integrally formed (e.g., by machining the optic and haptics together from a single block of material); and also the closed looped haptic IOLs. Yet a further style of IOL is called the plate haptic type wherein the haptics are configured as a flat plate extending from opposite sides of the optic. The IOL may be made from a variety of materials or combination of materials such as PMMA, silicone, hydrogels and silicone hydrogels, etc.

Various instruments and methods for implanting the IOL in the eye are known. In one method, the surgeon simply uses surgical forceps having opposing blades used to grasp the IOL and insert it through the incision into the eye. While this method is still practiced today, more and more surgeons are using more sophisticated IOL inserter devices which offer advantages such as affording the surgeon more control when inserting the IOL into the eye. IOL inserter devices have recently been developed with reduced diameter insertion tips which allow for a much smaller incision to be made in the cornea than is possible using forceps alone. Smaller incision sizes (e.g., less than about 3mm) are preferred over larger incisions (e.g., about 3.2 to 5+mm) since smaller incisions have been attributed to reduced post-surgical healing time and complications such as induced astigmatism.

Since IOLs are very small and delicate articles of manufacture, great care must be taken in their handling. In order for the IOL to fit through the smaller incisions, they need to be folded and/or compressed prior to entering the eye wherein they will assume their original unfolded/uncompressed shape. The IOL inserter device must therefore be designed in such a way as to permit the easy passage of the IOL through the device and into the eye, yet at the same time not damage the delicate IOL in any way. Should the IOL be damaged during delivery into the eye, the surgeon will most likely need to extract the damaged IOL from the eye and replace it with a new IOL, a highly undesirable surgical outcome.

Thus, as explained above, the IOL inserter device must be designed to permit easy passage of the IOL therethrough. It is equally important that the IOL be expelled from the tip of the IOL inserter device and into the eye in a predictable orientation and manner. Should the IOL be expelled from the tip too quickly or in the wrong orientation, the surgeon must further manipulate the IOL in the eye possibly resulting in trauma to the surrounding tissues of the eye. It is therefore highly desirable to have an inserter device which allows for precise loading of the IOL into the inserter device and which will pass and expel the IOL from the inserter device tip and into the eye in a controlled, predictable and repeatable manner.

To ensure controlled expression of the IOL through the tip of the IOL inserter device, the IOL must first be loaded into the IOL inserter device. The loading of the IOL into the inserter device is therefore a precise and very important step in the process. Incorrect loading of an IOL into the inserter device is oftentimes cited as the reason for a failed IOL delivery sequence. Many IOL inserter devices on the market today require the IOL to be loaded into the inserter at the time of surgery by the attending nurse and/or surgeon. Due to the delicate nature of the IOL, there is a risk that the nurse and/or surgeon will inadvertently damage the IOL and/or incorrectly load the IOL into the inserter device resulting in a failed implantation.

In a typical IOL inserter device, the IOL inserter utilizes a plunger having a tip which engages the IOL (which has been previously loaded and compressed into the inserter lumen) to pass it through the inserter lumen. The IOL thus interfaces with the plunger tip as well as the lumen of the inserter device. The lumen typically is dimensioned with a narrowing toward the open tip thereof in order to further compress the IOL as it is advanced through the lumen. The tip of the lumen is sized for insertion through the surgical incision which, as stated above, is presently preferred in the sub 3mm range. Thus, an inserter lumen will typically be dimensioned larger at the load area of the IOL and gradually decrease in diameter to the tip of the lumen where the IOL is expressed into the eye. It will be appreciated that the compressed diameter of the IOL at the lumen tip is the same as the inner diameter of the lumen tip, preferably sub 3 mm as stated above. Each of these component interfaces are dynamic in the sense that the forces acting between the interfacing components (i.e., the IOL, the plunger tip and the inserter lumen) will vary as the IOL is pushed through the lumen. Control of these dynamic forces is therefore of utmost importance or otherwise the IOL may be damaged during delivery due to excessive compressive forces acting thereon. For example, as the IOL is advanced by the plunger through an ever-decreasing diameter lumen, the IOL is being compressed while at the same time the forces necessary to push the IOL through the lumen increase. This may lead to excessive force between the plunger tip and the IOL resulting in possible damage to the IOL and/or uncontrolled release of the IOL from the lumen tip. Also, the force of the plunger tip may cause the IOL to twist and/or turn as it is moved through the inserter whereby the force between the IOL and the plunger tip and/or the inserter lumen may uncontrollably increase to the point of IOL damage.

Various inserter devices have been proposed which attempt to address these problems, and particularly the problem of controlling the force between the plunger, lumen and IOL, yet there remains a need for an IOL inserter which itself is simple in design and also simplifies operation of the IOL inserter device and thereby the IOL delivery process.

### SUMMARY OF THE INVENTION

The invention provides an IOL inserter having an improved plunger component for inserting IOLs having one or more curved haptics extending from the optic thereof. In particular, the plunger shaft incorporates a recessed area defined between the distal plunger tip and a curvilinear ramp wherein the trailing haptic of the IOL resides throughout the IOL delivery process. The curvilinear ramp acts as a stop for the trailing haptic whereby any chance of the haptic becoming entangled around the plunger shaft during IOL delivery is minimized or eliminated. In the preferred embodiment, the curve of the ramp closely matches the curve of the trailing haptic.

### BRIEF DESCRIPTION OF THE DRAWINGS

FIG. 1 is a perspective view of a prior art inserter device;

FIG. 2 is a fragmented, top plan view of the IOL loading area of the device of FIG. 1;

FIG. 3 is an enlarged, partial perspective view of the plunger component of FIGS. 1 and 2; and

FIG. 4 is an enlarged, partial perspective view of the inventive plunger component.

### DETAILED DESCRIPTION

### II. Prior Art

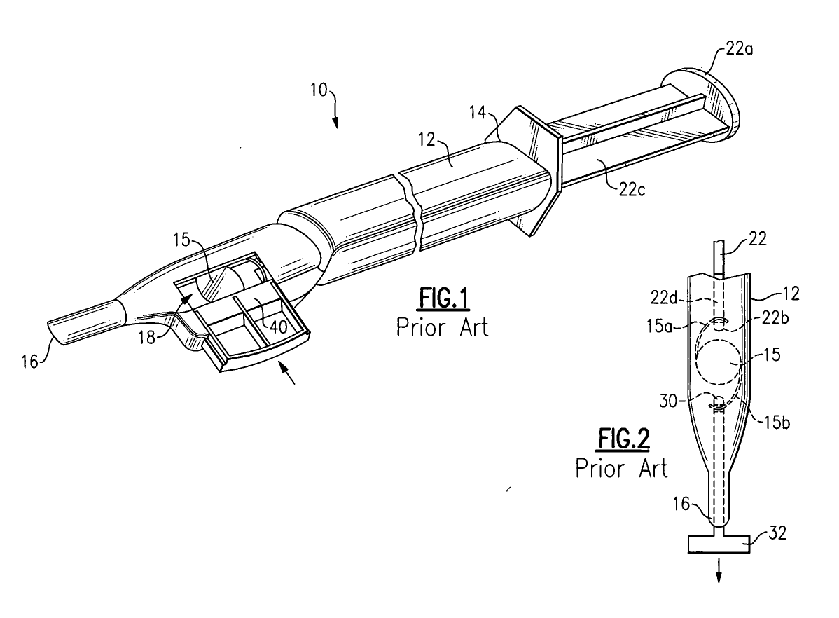

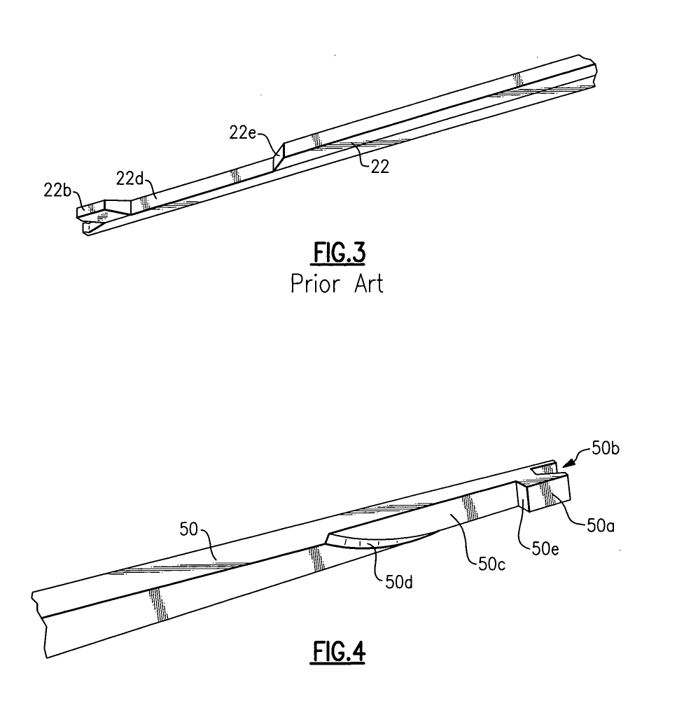

The present invention is directed towards IOL inserters having a tubular body defining a longitudinal passageway or lumen extending from a proximal end to a distal tip of the inserter, and a plunger component which telescopes in the open proximal end of the tubular body. A representative prior art inserter device 10 is seen in FIGS. 1-3 and is disclosed in commonly owned U.S. Pat. No. 5,944,725, the entirety of which is incorporated herein by reference. Device 10 includes a tubular body 12 having an open proximal end 14 and opposite distal tip 16 which is inserted into an incision in an eye for passing an IOL 15 through the inserter, out tip 16 and into the eye. IOL 15 is loaded into body 12 through any known IOL loading means, e.g., through an opening 18 designed into the inserter body. Body proximal end 14 includes a finger hold 20 and plunger 22 includes a finger press 22a at the proximal end thereof for pressing and advancing the plunger through the longitudinal passageway of the tubular body 12 in the manner of a syringe. A plunger tip 22b which may be bifurcated as seen best in FIG. 3, is provided at the distal end of the plunger for engaging and pushing the IOL optic 15 through and out the distal tip 16 of the inserter body 12. The IOL includes two curved haptics 15a and 15bas seen in FIG. 2. The IOL optic 15 is placed in the inserter body 12 with the proximal, trailing haptic 15a positioned within the plunger recess 22d and the distal, leading haptic 15bengaged by a catch 30 of haptic puller 32. Once the IOL has been initially positioned in body 12, compressor 40 is closed which compresses the IOL optic 15 within body 12. Haptic puller 32 is pulled away from inserter tip 16 which draws and straightens the leading haptic 15binto the inserter tip. This prevents the leading haptic from becoming bunched up within the inserter as the IOL is expressed therefrom and into an eye.

It is important that the plunger 22 be properly aligned and rotationally fixed within the tubular body so as to ensure proper engagement of the IOL 15 with plunger tip 22b. Plunger alignment and rotational fixing can be achieved by forming the body 12 and proximal end of the plunger in a non-circular (e.g., oval) cross-sectional shape. The proximal end of the plunger may be provided with a plurality of radially extending fins 22c which act to align and rotationally fix plunger 22 within the tubular body.

It is understood that the invention is directed at the plunger component and particularly the plunger-haptic interface thereof. The other parts of the plunger and inserter (e.g., the cross-sectional shape of the inserter body, the IOL loading area, the inserter distal tip, the plunger tip, etc.) may therefore be of any desired configuration. A prior art plunger 22 has a plunger-haptic interface in the form of a recess 22d. As seen best in FIG. 3, recess 22d is defined on the plunger shaft between distal tip 22b and stepped surface 22e. The stepped surface 22e is rather abrupt and includes sharp edges which could cause damage to the trailing haptic during delivery of the IOL through the inserter. Furthermore, surface 22e lies along a plane perpendicular to the longitudinal axis of the plunger shaft 22. This creates an unwanted stress point along the plunger shaft which could cause plunger failure, particularly when a high axial force is being applied to the plunger which may be necessary to push the IOL through a sub 3 mm inserter tip.

### II. A Preferred Embodiment of the Invention

A preferred embodiment of a plunger 50 is seen in FIG. 4. Plunger 50 may take the place of plunger 22 in inserter 10 although it is understood that plunger 50 may be used with other inserter designs as desired. Plunger 50 includes a plunger tip 50a which is preferably bifurcated to create slot 50b for engaging the periphery of the IOL optic 15 and push the IOL through inserter body 12 and out distal tip 16. A recess 50c is provided along the distal section of the plunger shaft and is defined by angled surface 50e adjacent plunger tip 50a at the distal end thereof and by a curvilinear ramp 50d at the other end thereof. The IOL trailing haptic 15a may be positioned within recess 50c when the IOL is initially loaded into the device in the manner shown in FIG. 2.

The curve of ramp 50d is preferably matches or at least approximates the curve of the trailing haptic such that the trailing haptic may lie in abutting relationship along the length of the ramp 50d. As such, ramp 50d acts as a stop against which the trailing haptic abuts during plunger advancement. This stop feature prevents the trailing haptic from becoming entangled around the plunger shaft during plunger advancement. Curvilinear ramp 50d traverses as a smooth arc across the plunger shaft and removes the sharp edged perpendicular orientation of the prior art stepped surface 22e. A such, the stress point created by the prior art recess configuration is not present in the preferred embodiment of the present invention and the potential for plunger failure caused by such a stress point is therefore minimized or eliminated.

In the preferred embodiment, the plunger tip is bifurcated as stated above and includes a slot 50b for engaging the periphery of the IOL optic 15. The plane along which slot 50b lies is therefore the same as the plane along which the IOL optic 15 lies. Since the trailing haptic initially extends along the plane of the optic, the plane along which curvilinear ramp 50d lies should likewise be parallel to the plane in which slot 50b lies. As such, proper orientation of the IOL optic and trailing haptic with respect to the plunger 50 is ensured.

1. An inserter for inserting an IOL having an optic with a peripheral edge and at least one curved haptic extending from said optic into an eye, said inserter comprising:
a) an inserter body having a proximal end and a distal tip and a longitudinal passageway extending therebetween;
b) a plunger for telescoping within said longitudinal passageway of said body, said plunger having a plunger shaft extending between a proximal end and a distal plunger tip, said plunger including a recess between said plunger tip and a curvilinear ramp formed in said plunger shaft;
whereby the at least one haptic of said IOL may be positioned in said recess during delivery through said inserter.

2. The inserter of claim 1 wherein the curve of said at least one haptic matches the curve of the said curvilinear ramp whereby the trailing haptic may engage said curvilinear ramp in abutting relationship thereto.

3. The inserter of claim 1 wherein said plunger tip is bifurcated and includes a slot for engaging the optic periphery.

4. The inserter of claim 3 wherein said curvilinear ramp extends along a plane which lies parallel to the plane in which said slot lies.

5. The inserter of claim 2 wherein said plunger tip is bifurcated and includes a slot for engaging the optic periphery.

6. The inserter of claim 5 wherein said curvilinear ramp extends along a plane which lies parallel to the plane in which said slot lies.

7. A plunger for telescoping and pushing an IOL through the longitudinal passageway of an IOL inserter and into an eye, said plunger having a plunger shaft extending between a proximal end and a distal plunger tip, said plunger including a recess between said plunger tip and a curvilinear ramp formed in said plunger shaft;
whereby a haptic of an IOL may be positioned in said recess during delivery of the IOL through the inserter.

8. The inserter of claim 7 wherein the curve of the IOL haptic matches the curve of the said curvilinear ramp whereby the haptic may engage said curvilinear ramp in abutting relationship thereto.

9. The inserter of claim 7 wherein said plunger tip is bifurcated and includes a slot for engaging the optic periphery.

10. The inserter of claim 8 wherein said curvilinear ramp extends along a plane which lies parallel to the plane in which said slot lies.

11. The inserter of claim 10 wherein said plunger tip is bifurcated and includes a slot for engaging the optic periphery.

12. The inserter of claim 9 wherein said curvilinear ramp extends along a plane which lies parallel to the plane in which said slot lies.
